(19) United States
(12) Plant Patent Application Publication
(10) Pub. No.: US 2024/0306520 P1
(43) Pub. Date: Sep. 12, 2024

(54) Hydrangea macrophylla 'HYD15'

(50) ;

(71) Applicant: Peter Fotinos, Santa Barbara, CA (US)

(72) Inventor: Peter Fotinos, Santa Barbara, CA (US)

(21) Appl. No.: 18/445,022

(22) Filed: Mar. 9, 2023

(51) Int. Cl. A01H 6/48

(52) U.S. Cl. CPC A01H 6/48
USPC PLT/250

(57) ABSTRACT

A new hydrangea macrophylla pink or blue with attractive flower. This variety propagates from soft wood cuttings and is stable in subsequent generations and is used for year-round production in greenhouse for flowering pot plant production.

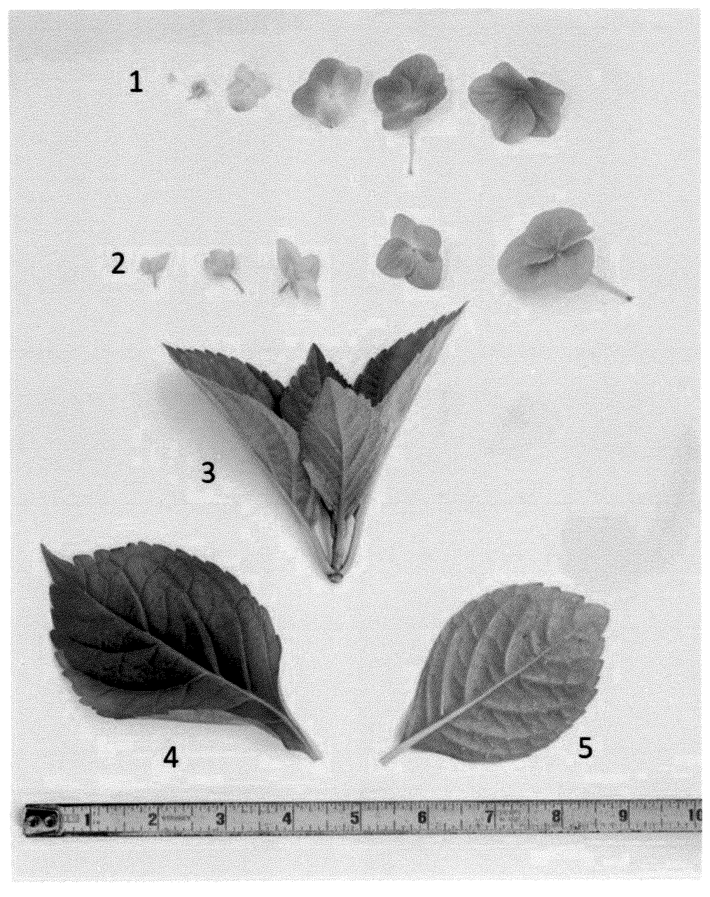

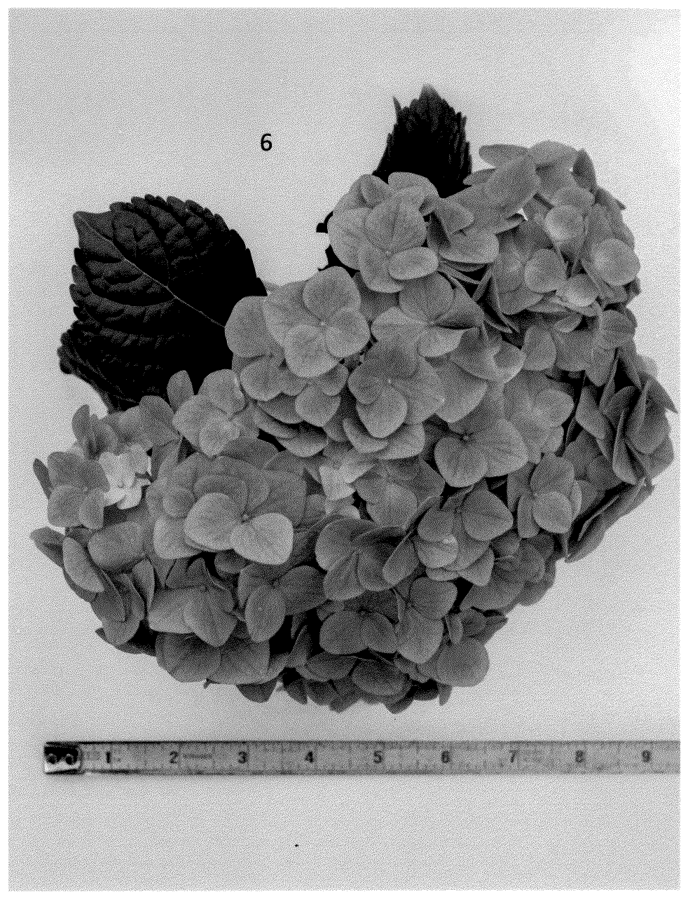

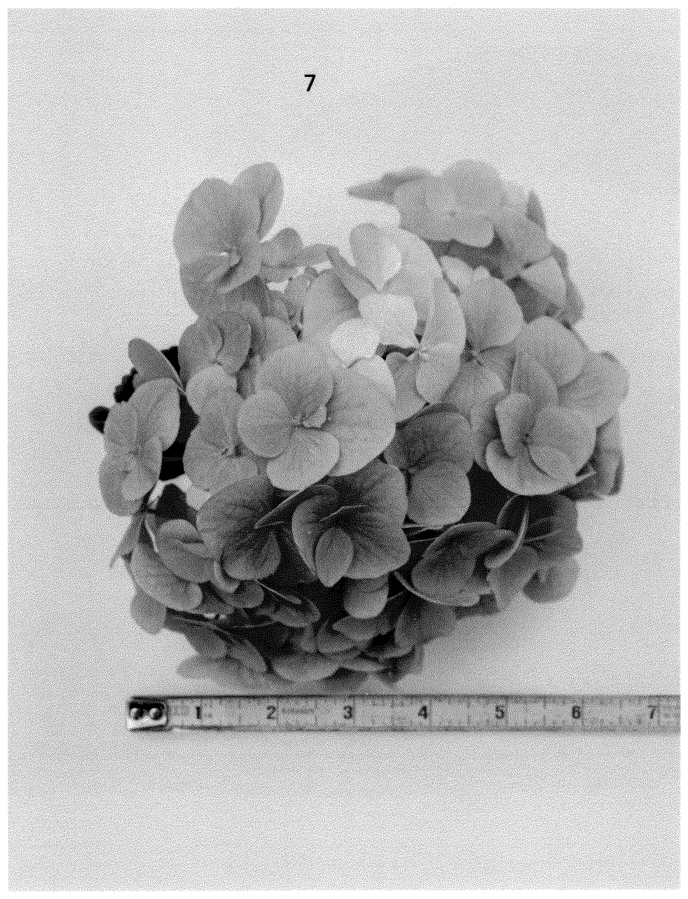

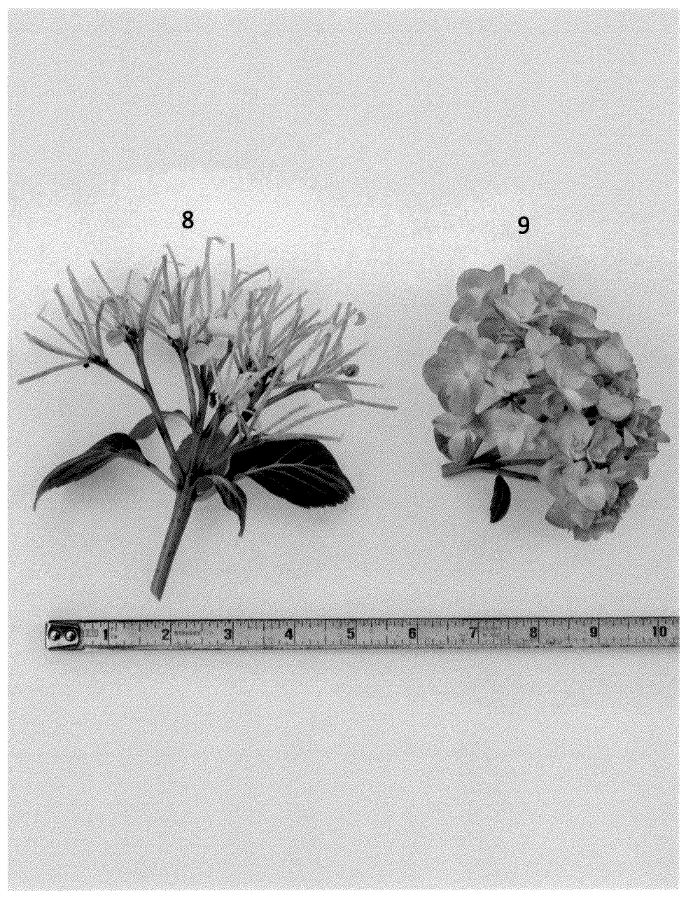

Latin name of genus and species: Hydrangea macrophylla ‘HYD15’

Variety denomination: the new variety is a Hydrangea plant named: ‘HYD15’.

### BACKGROUND OF THE INVENTION

The two Hydrangea macrophylla parents were crossed in the fall of 2016 to breed the new Hydrangea plant ‘HYD15’. The two parents were female unnamed seedling blue bicolor (not patented in the US) x male Mathilda Gutges blue (not patented in the US). The female parent was the unnamed seedling blue bicolor with blue and white flowers and large inflorescences. Mathilda Gutges is a named variety of hydrangea with dark blue flowers and large inflorescences. ‘HYD15’ has medium size inflorescences which are light blue or pink. It may be distinguished from its pollen parent by the lighter flower color, the faster finish time, and the more numerous flowers it has blooming at one time. It may be distinguished from the female parent by the color of its flower the more numerous flowers and the plant habit a full inflorescence. The plant was reproduced in Santa Barbara, CA in an indoor greenhouse environment.

### BRIEF SUMMARY OF THE INVENTION

Initial asexual reproduction was done of ‘HYD15’ by cuttings in Santa Barbara, California, U.S.A. The reproduction was conducted in a controlled greenhouse environment.

Have here proven that the foregoing and all after characteristics and distinctions come true to form and are established in succeeding propagations. Hydrangea ‘HYD15’ is a low vigorous compact hydrangea for pot production. The inflorescence bloom will last about 4-8 weeks in the container. The objective of the hybridization was to find a unique variety with:

1. Uniform and abundant flowers with good keepability for the floral industry

2. Attractive long-lasting foliage

3. Suitable for soft wood propagation

4. Remontant

5. Resistance to heat distortion during flower formation common with Dutch bred hydrangeas.

‘HYD15’ has proven to be all the above making flowers year-round in the greenhouse structure in Santa Barbara California with no distortion over the trial periods. ‘HYD15’ will rebloom within the same year without vernalization
We are not aware of any other varieties with these characteristics.

### BRIEF DESCRIPTIONS OF THE DRAWINGS

The accompanying color illustrations show as true as is reasonable to obtain in color photographs of this type, the typical characteristics of the buds, flowers leaves, stems of ‘HYD15’. The plant photographed below is 44-46 weeks old. The hydrangeas in photographs 1 and 6 are treated with aluminum sulfate to create a blue color. Specifically illustrated in the drawings:

On Sheet ¼
1: Development of blue flowers
2: Development of pink flower
3: Tip of young shoot
4: Upper side of leaf
5: Underside of leaf

On Sheet 2/4
6: Blue flower head fully unfolded.

On Sheet ¾
7: Pink flower head unfolded.

On Sheet 4/4
8: Flower bare corymb
9: Corymb starting to unfold.

### DETAILED BOTANICAL DESCRIPTION OF THE VARIETY

The following is a detailed description of the Hydrangea plant: Hydrangea macrophylla ‘HYD15’.

The following observations, measurements, values, and comparisons describe plants grown in glass houses in Santa Barbara, California, U.S.A.

The age of observed plants were 44 to 48 weeks old produced in a 10 cm diameter pot.

Environmental conditions were between 34-90 degrees Fahrenheit. Ten to twenty hours a day with assimilation light. Light levels of 600-6,000 foot candles. The relative humidity has been 50 to 85%. Plants have been regulated with PGR to modify plant shape and height.

Color References are using The Royal Horticultural Society (London, England) Colour Chart, 2019 (reprint). For comparison with existing hydrangea ‘Early Blue’ (‘200749077’, U.S. Plant Pat. No. 16,441 P2).

CHART 1

| Chart 1: | HYD15 | Early Blue |
|---|---|---|
| Sepal Color | 111A blue green veins 100B purple | Strong Purplish |
| Upper Surface | blue light reddish N78D or | Blue 95C |
| | deep purplish pink N57B to D | |
| Sepal Color | Moderate Purplish Pink 68 D | Violet blue group |
| Reverse | blue green group 111A | 98C light blue |
| Surface | venation 111D pale blue | |
| Sterile Flower | 200 to 270 | 150-200 |
| Count | | |
| Sepal Count | 4-7 | 4 |

Parents.—The two parents were crossed in the fall of 2016. The two parents were unnamed seedling blue bicolor and Mathilda Gutges blue.
Classification:
   Botanical.—Hydrangea macrophylla.
   Commercial.—Pot Hydrangea
Plant
   Plant growth.—Vigorous upright growth with compact habit when grown as a 11 cm average plant height is 25 to 30 cm average width is 20 cm. When grown in a 16 cm pot average height is 42 to 47 cm width is 30-35 cm. From unrooted cutting to finishing in either pot is 42 to 46 weeks depending on cultural practices and environmental conditions the habit is rounded. Production time is generally 44 to 48 weeks from unrooted cutting to finished plant depending on temperature light level and time of year. Pink flowers have soil pH over 6 and blue flowers are 4.5 pH to 5 pH with 20 ppm of aluminum sulfate added to the irrigation water.
Stem:
   Color.—Stem color young wood green group yellowish green 134C internode base color on stem red purple group strong purplish red 67A stem. Hard wood grey-brown group strong yellow brown 199D and darker areas with dark greyish yellow brown N199B.
   Lenticels.—On stems red purple group strong purplish red 67A 30-60 between nodes ovate vertical 0.3 to 0.5 mm.
   Surface.—Young Wood: Smooth Older Wood: Smooth.
   Stem diameter.—Diameter 4-5 mm.
   Internode length.—20-50 mm.
   Number of internodes.—2 to 6.
Plant foliage: Leaves arranged symmetrically abundant and flat in aspect. The venation pattern is pinnate.
   Quantity of leaves.—4 to 10 leaves per stem.
   Petiole.—Color: yellow green group 150D 3 to 4 mm.
   Leaves.—Pinnate venation leaves serrated edge single shape ovate with acuminate apex and obtuse to acuminate upper leaf texture coriaceous shiny surface length of leaf is 4 cm to 11 cm width is 5 cm to 8 cm. Lower leaf texture is soft and coriaceous. Leaf color upper green group dark yellowish green 139A venation moderate yellowish green 139C. Under the leaf strong yellow green 143B strong yellow green 143A venation edges are serrated.
Inflorescence:
   Blooming habit.—Seasonal late spring through summer.
   Fragrance.—None.
   Flower type.—Single flowers densely arranged on a corymb spherical formed head.
   Flower heads size.—Diameter of 12 cm to 18 cm and height of 7 cm to 13 cm. Sterile flowers have longer pedicles than fertile flowers. Sterile flowers are above fertile flowers and fertile flowers usually senesce before the sterile flowers are completely open. There are 30 to 50 fertile flowers when the flower is just starting to open and have 0 fertile flowers at the end of sterile flower development. Fertile flowers are 1 to 2 mm diameter height 1-2 mm shape ovoid.
   Peduncle.—Moderate yellowish green 139C.
   Pedicel.—Group yellowish green 134C smooth 6 to 12 pedicel per peduncle erect/strong end of pedicel light purplish pink 63D 1 to 2 mm diameter 2.5 to 3.8 cm smooth length.
   Sterile flower.—Yellow green group moderate 139C: at opening cup when viewed from the side 4-7 sepals per flower obtuse shape with some toothing midway 3:0-3.8 cm width 3.0 to 4.0 cm length 0.200 to 270 sterile flowers per corymb. Ph of 7 color group Sepal color pink: red purple group deep purplish pink N57B veins young flowers strong yellow green 145B in center color group reverse side is red purple group 68D getting lighter as we get to center when young to green white group pale yellow green 157A at center. 5.5 pH Sepal color upper blue: blue green group: 111A veins 100B purple group light reddish purple N78D 4 to 5 sepals. Sepal color under blue: blue green group 111A venation 111D pale blue Changing from pink to blue the colors of sepal vary from violet group 84C to 84A strong purplish blue 98D to 98A Center 97B light purplish blue.
Development:
   Vegetation.—Dense.
   Blooming.—Abundant.
   Aptitude to bear fruit.—Poor.
   Resistance to diseases.—Above normal disease resistance: Powdery and Downy Mildew, Pythium, Botrytis in Santa Barbara growing conditions.
   Winter hardiness & drought/heat tolerance.—Not tested for winter hardiness. Drought tolerance.

The invention claimed is:

1. A new and distinct variety of Hydrangea plant named ‘HYD15’, will develop flowers year-round under short day conditions at a temperature of 52 F. to over 90 F. (11. C. to 32 C.), the variety is remontant, which means it will bloom again after the first flower is cut off without vernalization.
